(19) United States
(12) Patent Application Publication
(10) Pub. No.: US 2014/0140042 A1
(43) Pub. Date: May 22, 2014

(54) IMITATION CANDLE

(71) Applicant: Daniel SCHREIBER, Concord, CA (US)

(72) Inventor: Daniel SCHREIBER, Concord, CA (US)

(21) Appl. No.: 13/681,514

(22) Filed: Nov. 20, 2012

(51) Int. Cl. A61L 9/03

(52) U.S. Cl. CPC A61L 9/037
USPC 362/96

(57) ABSTRACT

An imitation candle comprises an imitation candle body, an electric light source supported by the imitation candle body for producing an imitation flame, and a fragrance emitter supported by the imitation candle body for producing a fragrance.

### FIELD

The disclosure relates to an imitation candle including an electric light source and a fragrance emitter.

### BACKGROUND

U.S. Pat. No. 6,616,308 (Jensen et al.) purports to disclose an imitation candle made from a translucent material having light transmissive properties similar to paraffin. The imitation candle is shaped to appear reduced by burning. An LED, or similar high intensity light source, is set in a cavity within the imitation candle. The LED preferably produces amber light to better resemble the color of candle light. The imitation candle diffuses the light emitted from the LED to create a warm, natural looking glow. Light emission levels from the LED are varied in a pseudo-random manner to simulate the flicker of candle light.

U.S. Pat. No. 6,719,443 (Gutstein et al.) purports to disclose an electrically powered flame simulator comprising at least two light sources. An integrated circuit is electrically connected to the light sources for intermittently illuminating at least one of the light sources independently of other light sources such that the light sources together provide the effect of a flickering movement. A power source provides power to the integrated circuit. The flame simulator may be mounted in a decorative or ornamental device such as a candle or fire log, or used on decorative clothing, or may be part of a hazard or warning system. One or more solid state light sources may also be used.

U.S. Pat. No. 7,118,243 (McCavit et al.) purports to disclose an imitation candle having an exterior wax shell and an interior illumination source. The imitation candle is structured to reduce cracking of the exterior wax shell by incorporating a bonding layer into a central cavity in the shell between the interior illumination source and the wax shell. The bonding layer terminates well-spaced from terminal edges of the shell to transfer stress between the illumination source and the shell caused by differing coefficients of thermal expansion to points removed from edges of the shell to retard crack genesis.

### SUMMARY

The following summary is intended to introduce the reader to various aspects of the disclosure, but not to define any invention.

According to one aspect, an imitation candle comprises an imitation candle body, an electric light source supported by the imitation candle body for producing an imitation flame, and a fragrance emitter supported by the imitation candle body for producing a fragrance.

In some examples, the fragrance emitter may comprise a fragrance reservoir positioned within the imitation candle body for storing a fragrant fluid. The fragrance emitter may further comprise a fragrance wick coupled to the fragrance reservoir for diffusing the fragrance from the fragrant fluid. At least one of the fragrance reservoir and the fragrance wick may be removable from the imitation candle body. The fragrance emitter may further comprise an electric heat source for heating the fragrant fluid to actively diffuse the fragrance from the fragrant fluid. The electric heat source may comprise a heating ring proximate the fragrance wick.

In some examples, the imitation candle may further comprise a power source housed within the imitation candle body and powering the electric light source and the fragrance emitter.

In some examples, the candle body may comprise a generally cylindrical outer wall defining an interior cavity, and an interior wall extending across the interior cavity transversely to the outer wall. The interior wall may divide the interior cavity into an upper cavity and a lower cavity. The upper cavity may be generally open to the outside environment, and the lower cavity may comprise a bottom cover extending thereacross and generally closing the lower cavity to the outside environment. At least a portion of the bottom cover may be openable to provide access to the fragrance reservoir. The portion of the bottom cover may comprise a first openable door, and the fragrance emitter may comprise a fragrance reservoir removable from the lower cavity through the first openable door.

In some examples, the imitation candle may further comprise a power source housed within the lower cavity and powering the electric light source and the electric heat source. At least a portion of the bottom cover may be openable to provide access to the power source. The portion of the bottom cover may comprise a second openable door, and the power source may be removable from the lower cavity through the second openable door.

In some examples, the electric light source may be positioned above the interior wall in the upper cavity.

In some examples, the fragrance emitter may comprise a fragrance reservoir, and the fragrance reservoir may be positioned in the lower cavity. The interior wall may comprise a fragrance aperture proximate the fragrance reservoir and extending between the upper cavity and the lower cavity. The fragrance emitter further may comprise a fragrance wick coupled to the fragrance reservoir for disseminating a fragrance from the fragrance reservoir, and the fragrance wick may extend from the fragrance reservoir and into the fragrance aperture.

In some examples, the imitation candle may comprise a power source powering the electric light source and the electric heat source, and the power source may be housed within the lower cavity.

In some examples, the electric light source may comprise a light emitting diode.

According to another aspect, an imitation candle comprises an imitation candle body defining an upper cavity generally open to the outside environment, and a lower cavity. The imitation candle further comprises an electric light source for producing an imitation flame. The electric light source is positioned in the upper cavity. The imitation candle further comprises a fragrance emitter for producing a fragrance. The fragrance emitter comprises (i) a fragrance reservoir for storing a fragrant fluid and positioned in the lower cavity, (ii) a fragrance wick coupled to the fragrance reservoir for diffusing a fragrance from the fragrant fluid, and (iii) an electric heat source for heating the fragrant fluid. A power source is positioned in the lower cavity and powers the electric light source and the electric heat source.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1A is a top perspective view of an example of an imitation candle;

FIG. 1B is a bottom perspective view of the imitation candle of FIG. 1A;

FIG. 2 is a front plan view of the imitation candle of FIG. 1A;

FIG. 3 is a cross-sectional view taken along line 3-3 in FIG. 2;

FIG. 4 is an exploded view of the imitation candle of FIG. 1A;

FIG. 5 is a perspective view of an insert of the imitation candle of FIG. 1A, showing several internal components of the imitation candle; and

FIG. 6 is a bottom view of the insert of FIG. 5.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any exclusive right granted by issuance of this patent application, Any invention disclosed in an apparatus or process described below and for which an exclusive right is not granted by issuance of this patent application may be the subject matter of another protective instrument, for example, a continuing patent application, and the applicants, inventors or owners do not intend to abandon, disclaim or dedicate to the public any such invention by its disclosure in this document.

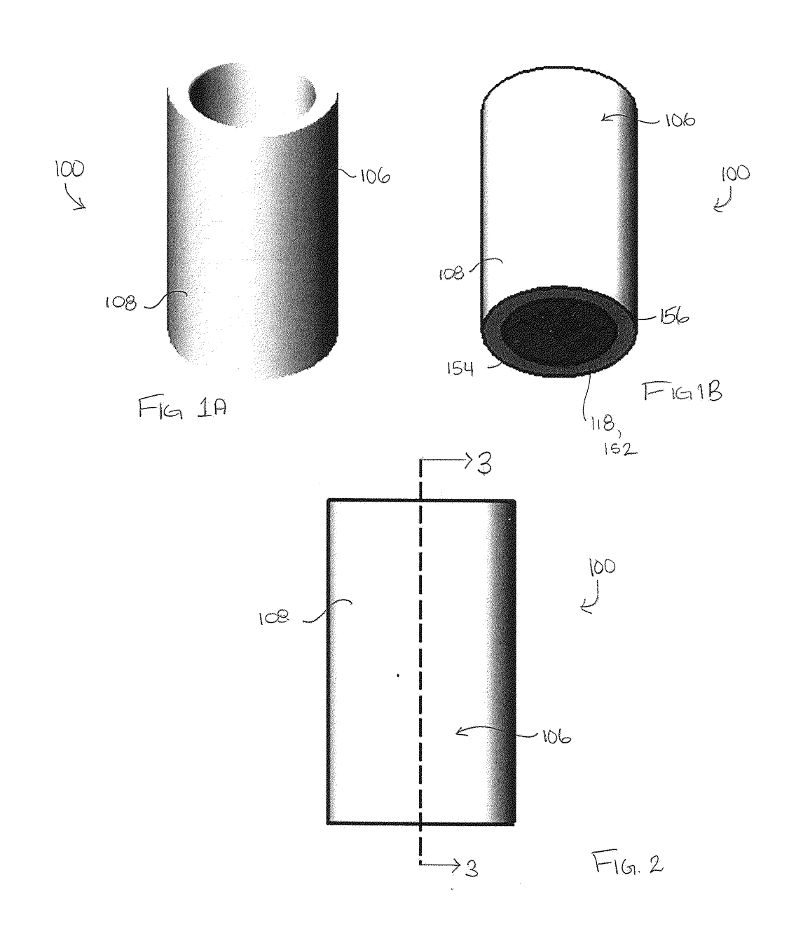

Referring to FIGS. 1A to 3, an example imitation candle 100 (also referred to as ‘candle 100’) is shown. The candle 100 is without a real flame, and generally includes an electric light source 102 for producing an imitation flame, and a fragrance emitter 104 for producing a fragrance. The candle further includes an imitation candle body 106 (also referred to as ‘body 106’), which supports the light source 102 and the fragrance emitter 104. The body 106 is generally shaped to have the appearance of a candle. Together, the body 106, imitation flame, and fragrance provide the ‘feel’ or impression of a real candle.

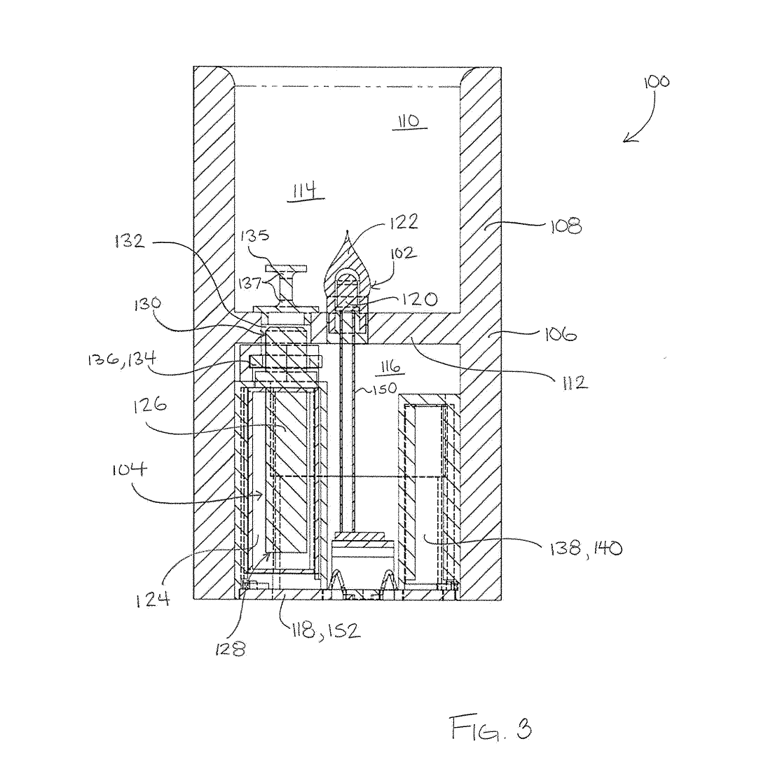

Referring to FIGS. 1A to 2, in the example shown, the body 106 is shaped to have the appearance of a cylindrical pillar candle. In other examples, the body may be shaped to have the appearance of another type of candle, including but not limited to a pillar candle of another shape (e.g. cubic, spherical, or otherwise), a tealight, a votive candle, a container candle, or a taper candle.

The body 106 may be fabricated from a wax or another material, such as one that has an appearance similar to wax. Examples of materials include, but are not limited to, plastics, glass such as frosted glass, wood, or fabric. The body 106 may optionally be translucent, to allow for some light from the electric light source to pass therethrough.

Referring still to FIGS. 1A to 3, in the example shown, the body 106 includes a generally cylindrical outer wall 108 defining an interior cavity 110, and an interior wall 112 extending across the interior cavity 110 transversely to the outer wall 108. The interior wall 112 divides the interior cavity into an upper cavity 114 and a lower cavity 116. The upper cavity 114 is generally open to the outside environment. The lower cavity 116 includes a bottom cover 118, which generally closes the lower cavity 116 to the outside environment. As will be described in further detail below, at least a portion of the bottom cover 118 may be openable.

Referring now to FIG. 3, in the example shown, the electric light source 102 includes a light emitting diode (LED) 120, and a decorative element 122. The decorative element 122 generally has the appearance of a flame, and is mounted over the LED 120. The electric light source 102 is positioned in the upper cavity 114, just above the interior wall 112, so that it appears to rest on the interior wall 112. This positioning may provide the appearance of a candle that has burned down to some degree.

Other examples of electric light sources include, but are not limited to, incandescent light bulbs, neon light bulbs, or compact fluorescent bulbs.

Referring still to FIG. 3, in the example shown, the fragrance emitter 104 includes a fragrance reservoir 124, and a fragrance wick 126. The fragrance reservoir 124 is positioned in the lower cavity 116, and may store a fragrant substance, for example a fragrant fluid such as a volatile scented oil, or a gel. The fragrance wick 126 is coupled to the fragrance reservoir 124 and may aid in diffusing or disseminating the fragrance from the fragrant fluid in the fragrance reservoir 124. The fragrance wick 126 may be, for example, a cotton cord or a foam or sponge-like cylinder. The fragrance wick 126 may extend from a first end 128 within the fragrance reservoir, to a second end 130 within or proximate the upper cavity 114. In the example shown, the interior wall 112 includes a fragrance aperture 132 proximate the fragrance reservoir 124 and extending between the upper cavity 114 and lower cavity 116. The first end 128 is positioned within the fragrance reservoir 124, so that it is immersed in the fragrant fluid. The second end extends into and is positioned within the fragrance aperture 132.

As will be described in further detail below, the fragrance reservoir 124, together with the fragrance wick 126, may be removable from the lower cavity 116, for example so that the fragrance reservoir may be replaced when spent, or so that the fragrance reservoir 124 may be replaced with a fragrance reservoir containing a different scent.

In the example shown, the fragrance emitter 104 further includes a wick cover 135. The wick cover 135 is mounted to the interior wall 112 over the second end 130 of the fragrance wick 126, and includes ports 137 to allow the fragrance to be emitted.

The fragrance emitter 104 may further include an electric heat source for heating the fragrant fluid, in order to facilitate the emission of a fragrance from the candle 100 (i.e. to actively diffuse or disseminate the fragrance from the fragrant fluid). The electric heat source may heat the fragrant fluid directly, or indirectly by heating the fragrance wick 126 and/or the fragrance reservoir 124. Referring now to FIG. 3, in the example shown, the fragrance emitter 104 includes an electric heat source 134 for heating the fragrance wick 126. The electric heat source 134 includes a heating ring 136 proximate the fragrance wick 126 and extending around the fragrance wick 126. The heating ring 136 may be, for example, a copper wire or a ceramic ring. In other examples, the electric heat source may be of another configuraton other than a ring.

In some examples (not shown), the electric light source 102 and/or the electric heat source 134 may be powered by an external power source. For example, the candle 100 may plug into an electrical outlet to power the electric light source 102 and/or the electric heat source 134. In the example shown, the electric light source 102 and the electric heat source 134 are powered by an internal power source. Specifically, referring to FIG. 3, the electric light source 102 and the electric heat source 134 are powered by a power source 138 that is housed within the lower cavity 116 of the body 106. The power source 138 includes a pair of batteries 140, which may be rechargeable or disposable. The electric light source 102 and electric heat source 134 may be connected to the batteries 140 by standard wiring (not shown).

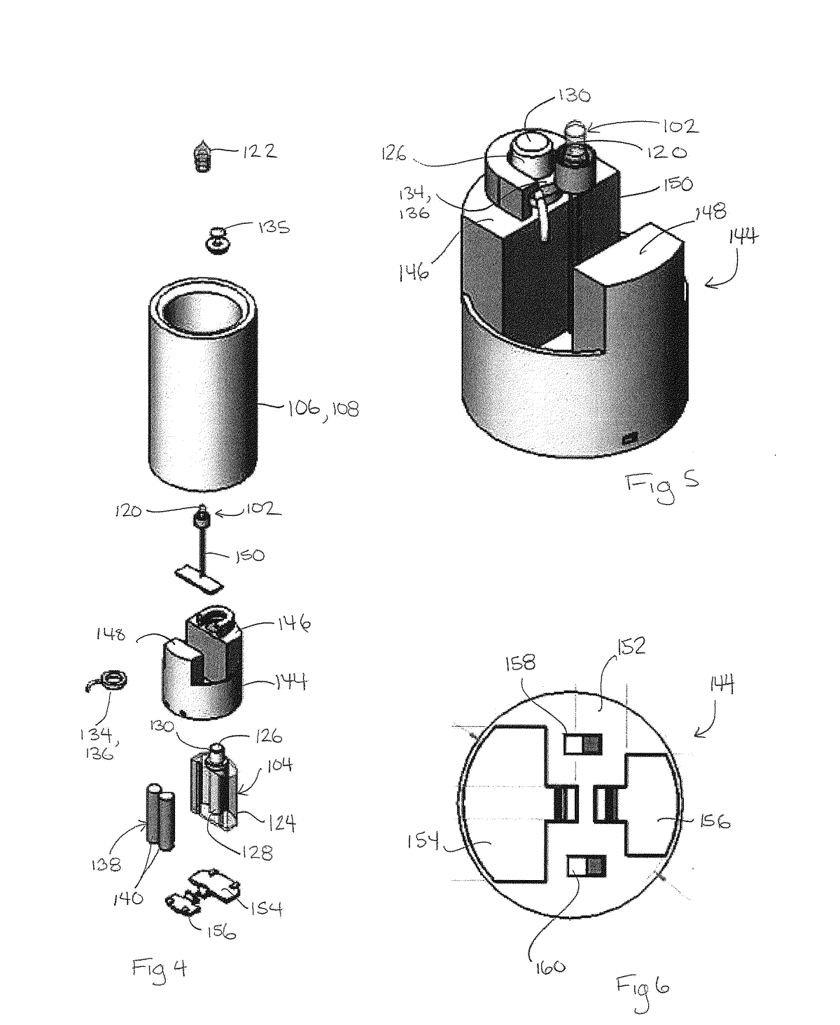

Referring now to FIGS. 4 and 5, the electric light source 102, fragrance emitter 104, and power source 138 are mounted to an insert 144 that is received in the lower cavity 116. The insert 144 includes a first compartment 146 in which the fragrance reservoir is received, and a second compartment 148 in which the batteries 140 are received. The electric light source 102 is mounted to a support 150, which is seated on the insert 144.

The insert 144 may be removable from or generally irremovable from the lower cavity 116 (the term ‘irremovable’ indicates that a consumer may not readily remove the insert from the lower cavity 116 without destroying the candle or a portion of the candle). In the example shown, the insert 144 is generally irremovable from the lower cavity 116, and includes a locking mechanism that engages the body 106 to irremovably secure the insert 144 to the body 106.

Referring still to FIGS. 4 and 5, and also to FIG. 6, in the example shown, the insert 144 includes a bottom wall 152, which forms the bottom cover 118 of the lower cavity 116. At least a portion of the bottom wall 152 may be openable to provide access to the fragrance reservoir 124. In the example shown, the bottom wall 152 includes a first openable door 154, which opens the first compartment 146 to provide access to the fragrance reservoir. The first openable door 154 may be, for example, pivotably openable. The fragrance reservoir 124, together with the fragrance wick 126, may be slidably removed from the lower cavity 116 through the first openable door 154. As mentioned above, the fragrance reservoir 124 may be removed so that it may be replaced.

In some examples (not shown), the exterior surface of the fragrance reservoir and the interior surface of the first compartment 146 may be keyed, so that only certain fragrance reservoirs may be inserted into the first compartment 146.

Referring still to FIGS. 4 to 6, the bottom wall 152 includes a second openable door 156, which opens the second compartment 148 to provide access to the batteries 140. The second openable door 156 may be, for example, pivotably openable. The batteries 140 may be slidably removed from the lower cavity 116 through the second openable door 156, for example so that they may be replaced.

The candle 100 may further include one or more controls for controlling the electric light source and/or the electric heat source. Referring to FIGS. 1B and 6, in the example shown, the candle 100 includes a first switch 158, and a second switch 160. The first switch 158 may be actuated by a user to turn the electric light source 102 on and off. The second switch 160 may be actuated by a user to turn the electric heat source 134 on and off.

In other examples (not shown), the candle may include a single switch, which may control both the electric light source and the electric heat source. In yet other examples, the candle may include one or more slides rather than switches. Slides may be used so that the intensity of the light and/or scent may be controlled by the user.

While the above description provides examples of one or more processes or apparatuses, it will be appreciated that other processes or apparatuses may be within the scope of the accompanying claims.

1. An imitation candle comprising:
a) an imitation candle body;
b) an electric light source supported by the imitation candle body for producing an imitation flame; and
c) a fragrance emitter supported by the imitation candle body for producing a fragrance.

2. The imitation candle of claim 1, wherein the fragrance emitter comprises a fragrance reservoir positioned within the imitation candle body for storing a fragrant fluid.

3. The imitation candle of claim 2, wherein the fragrance emitter further comprises a fragrance wick coupled to the fragrance reservoir for diffusing the fragrance from the fragrant fluid.

4. The imitation candle of claim 3, wherein at least one of the fragrance reservoir and the fragrance wick are removable from the imitation candle body.

5. The imitation candle of claim 3, wherein the fragrance emitter further comprises an electric heat source for heating the fragrant fluid to actively diffuse the fragrance from the fragrant fluid.

6. The imitation candle of claim 5, wherein the electric heat source comprises a heating ring proximate the fragrance wick.

7. The imitation candle of claim 5, further comprising a power source housed within the imitation candle body and powering the electric light source and the electric heat source.

8. The imitation candle of claim 1, wherein the imitation candle body comprises a generally cylindrical outer wall defining an interior cavity, and an interior wall extending across the interior cavity transversely to the outer wall, the interior wall dividing the interior cavity into an upper cavity and a lower cavity.

9. The imitation candle of claim 8, wherein the upper cavity is generally open to the outside environment, and the lower cavity comprises a bottom cover extending thereacross and generally closing the lower cavity to the outside environment.

10. The imitation candle of claim 9, wherein at least a portion of the bottom cover is openable to provide access to the fragrance emitter.

11. The imitation candle of claim 10, wherein the portion of the bottom cover comprises a first openable door, and the fragrance emitter comprises a fragrance reservoir removable from the lower cavity through the first openable door.

12. The imitation candle of claim 9, further comprising a power source housed within the lower cavity and powering the electric light source and fragrance emitter, wherein at least a portion of the bottom cover is openable to provide access to the power source.

13. The imitation candle of claim 12, wherein the portion of the bottom cover comprises a second openable door, the power source removable from the lower cavity through the second openable door.

14. The imitation candle of claim 8, wherein the electric light source is positioned above the interior wall in the upper cavity.

15. The imitation candle of claim 8, wherein the fragrance emitter comprises a fragrance reservoir, and the fragrance reservoir is positioned in the lower cavity.

16. The imitation candle of claim 15, wherein the interior wall comprises a fragrance aperture proximate the fragrance reservoir and extending between the upper cavity and the lower cavity.

17. The imitation candle of claim 16, wherein:
a) the fragrance emitter further comprises a fragrance wick coupled to the fragrance reservoir for disseminating a fragrance from the fragrance reservoir b) the fragrance wick extends from the fragrance reservoir and into the fragrance aperture.

18. The imitation candle of claim 8, further comprising a power source powering the electric light source and the fragrance emitter, the power source housed within the lower cavity.

19. The imitation candle of claim 1, wherein the electric light source comprises a light emitting diode.

20. An imitation candle comprising:
a) an imitation candle body defining an upper cavity generally open to the outside environment, and a lower cavity;
b) an electric light source for producing an imitation flame, the electric light source positioned in the upper cavity; and
c) a fragrance emitter for producing a fragrance, the fragrance emitter comprising: (i) a fragrance reservoir for storing a fragrant fluid, the fragrance reservoir positioned in the lower cavity, (ii) a fragrance wick coupled to the fragrance reservoir for diffusing a fragrance from the fragrant fluid, and (iii) an electric heat source for heating the fragrant fluid; and
d) a power source positioned in the lower cavity and powering the electric light source and the electric heat source.
